(19) United States
(12) Patent Application Publication
(10) Pub. No.: US 2009/0186015 A1
(43) Pub. Date: Jul. 23, 2009

(54) MICRORNAS DIFFERENTIALLY EXPRESSED IN LUNG DISEASES AND USES THEREOF

(76) Inventors: Gary J. Latham, Austin, TX (US); Heidi J. Peltier, Austin, TX (US)

Correspondence Address: Fullbright & Jaworski L.L.P., 600 Congress Avenue, Suite 2400, Austin, TX, 78701, US

(21) Appl. No.: 12/253,718

(22) Filed: Oct. 17, 2008

Related U.S. Application Data
(60) Provisional application No. 60/981,057, filed on Oct. 18, 2007.

(51) Int. Cl. A61K 39/395; C12Q 1/68

(52) U.S. Cl. 424/130.1; 435/6

(57) ABSTRACT

The present invention concerns methods and compositions for identifying a miRNA profile for a particular condition, such as lung cancer, and using the profile in assessing the condition of a patient.

This application claims priority to U.S. Provisional Application Ser. No. 60/981,057, filed Oct. 18, 2007, which is incorporated herein by reference in its entirety.

### BACKGROUND OF THE INVENTION

I. Field of the Invention

The present invention relates generally to the field of molecular biology. More particularly, it concerns methods and compositions involving microRNA (miRNAs) molecules. Certain aspects of the invention include applications for miRNAs in diagnostics, therapeutics, and prognostics for lung cancer.

II. Background

In 2001, several groups used a cloning method to isolate and identify a large group of miRNAs from C. elegans, Drosophila, and humans (Lagos-Quintana et al., 2001; Lau et al., 2001; Lee and Ambros, 2001). Several hundred miRNAs have been identified in plants and animals—including humans—which do not appear to have endogenous small interfering RNAs or silencing RNAs (siRNAs). Thus, while similar to siRNAs, miRNAs are distinct.

miRNAs thus far observed have been approximately 21-22 nucleotides in length, and they arise from longer precursors, which are transcribed from non-protein-encoding genes. See review of Carrington et al. (2003). The precursors form structures that fold back on themselves in self-complementary regions; they are then processed by the nuclease Dicer (in animals) or DCL1 (in plants) to generate the short double-stranded miRNA. One of the miRNA strands is incorporated into a complex of proteins called the RNA-induced silencing complex (RISC). The miRNA guides the RISC complex to a target messenger RNA (mRNA), which is then cleaved or translationally silenced, depending on the degree of sequence complementarity of the miRNA to its target mRNA. Currently, it is believed that perfect or nearly perfect complementarity leads to mRNA degradation, as is most commonly observed in plants. In contrast, imperfect base pairing, as is primarily found in animals, leads to translational silencing. However, recent data suggest additional complexity (Bagga et al., 2005; Lim et al., 2005), and mechanisms of gene silencing by miRNAs remain under intense study.

Other recent studies have shown that expression levels of numerous miRNAs are associated with various cancers (reviewed in Esquela-Kerscher and Slack, 2006; Calin and Croce, 2006), including lung cancer (Volinia et al., 2006; Yanaihara et al., 2006). miRNAs have also been implicated in regulating cell growth and cell and tissue differentiation—cellular processes that are associated with the development of cancer.

Lung cancer is the leading cause of cancer death worldwide in both men and women and is primarily caused by tobacco smoking. In 2007, approximately 160,000 people will die of lung cancer in the United States alone, with an estimate of 213,000 new cases (Jemal et al., 2007). Lung cancers are generally divided into two types (non-small cell lung cancers and small cell lung cancers). Non-small cell cancers are more common and include squamous cell carcinoma, adenocarcinoma, large cell carcinoma, adenosquamous carcinoma, and undifferentiated carcinoma. Among these, squamous cell carcinomas and adenocarcinomas are the most common types.

It is important to determine the type of lung cancer, as different carcinoma types involve different regions of the lung and are treated differently. Identification of a specific lung cancer type is usually initiated by an examination of cells in sputum (sputum cytology) followed by examination of cells collected from the lung or bronchus, usually by flexible bronchoscopy or fine needle aspiration (FNA). Once a tissue diagnosis has been established, a thorough staging analysis, including metastatic evaluation and staging classification, is frequently performed. miRNAs identified and utilized in the present invention for evaluating lung and lung diseases include, but are not limited to miR-221, miR-93, and miR-195.

U.S. Patent Publication 2005/0261218 describes methods and compositions for modulating small non-coding RNAs, including miR-221 and miR-93. The '218 publication is silent regarding expression levels of miR-221 or miR-93 and does not correlate such expression with diagnostics involving lung cancer.

U.S. Patent Publication 20070065844 describes analysis of expression levels of miR-221 and concludes that miR-221 is over expressed in normal tissue as compared to cancer samples evaluated. This description of miR-221 expression contradicts the evaluation provided in the present application.

Galardi et al. (2007) describe the elevated expression of miR-221 in aggressive prostate carcinomas. Galardi et al. only speaks to the association between miR-221 and its oncogenic effects in prostate cancer. Of note, Galardi et al. report detecting miR-221 overexpression predominantly in an aggressive prostate carcinoma cell and detected little if any elevated expression in other prostate cancer cell lines.

Yanaihara et al. (2006) assess miRNA profiles related to diagnosis and prognosis of lung cancer. Several miRs, not including miR-93, were identified as possible diagnostic miRs. Other miRs, including miR-93, were identified as correlating to patient survival/prognosis. Diagnosing and prognosing are two distinct undertakings in as much as diagnosing identifies a disease state, and prognosing includes many factors related to patient survival, disease progression, and outcome.

Volinia et al. (2006) describe the identification of miRNA expression signatures that include the elevated expression of miR-195 in breast, colon, lung, pancreas, prostate and stomach cancer. These findings are contradictory to the expression levels of miR-195 in lung cancer presented in the present application.

A need exists for additional diagnostic assays that can assess the cancerous state of lung cells in general and accurately distinguish cancerous cells or tissue from non-cancerous cells or tissue in particular.

### SUMMARY OF THE INVENTION

The present invention overcomes these problems in the art by identifying miRNAs that are differentially expressed or mis-regulated in various states of diseased, normal, cancerous, and/or abnormal tissues, including but not limited to normal lung and lung cancer (e.g., non-small cell lung cancer, small cell lung cancer, squamous cell carcinoma, adenocarcinoma, large cell carcinoma, adenosquamous carcinoma, or undifferentiated carcinoma). Further, the invention describes a method for diagnosing lung cancer that is based on determining levels (increased or decreased) of selected miRNAs in patient-derived samples. Certain genes and their regulatory pathways represent targets for therapeutic intervention by regulating their expression with miRNAs.

The term “miRNA” or “miR” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor. Names of miRNAs and their sequences related to the present invention are provided herein.

miR sequences can be used to evaluate lung tissue for the possibility of a hyperproliferative condition of the lung that is characterized by the presence of uncontrolled or hyperactive cell division that results in disease or a pathological condition. Hyperproliferative conditions include lung cancer, an invasive and/or metastatic hyperproliferative condition, and precancers, a self limiting hyperproliferative condition that, if left untreated, may lead to cancer. miR sequences include nucleic acids that comprise all or part of one or more of:

miR-221—(stem loop sequence UGAACAUCCAGGUCUGGGGCAUGAA CCUGGCAUACAAUGUAGAUUUCUGUGUUCGUUAGGCAACAGCUACAUUGU CUGCUGGGUUUCAGGCUACCUGGAAACAUGUUCUC (SEQ ID NO:1) and mature sequence AGCUACAUUGUCUGCUGGGUUUC (SEQ ID NO:2) and family consensus sequence of AGCUACAU(C/-)UG(U/G)CU(G/A)CUGGGU(U/C/-)(U/-)(C/-) (SEQ ID NO:3)).

miR-93—(stem loop sequence CUGGGGGCUCCAAAGUGCUGUU CGUGCAGGUAGUGUGAUUACCCAACCUACUGCUGAGCUAGCACUUCCCGA GCCCCCGG (SEQ ID NO:4) and mature sequence CAAAGUGCUGUUCGUGCAGGUAG (SEQ ID NO:5) and family consensus sequence (C/A/-)(A/C/-)AA(G/C)UGCUG(U/A)(U/G)C(G/U)(U/A)GCA(G/C)(G/U)U(A/C/-)(G/C/-)(-/C)(-/G) (SEQ ID NO:6)).

miR-195—(stem loop AGCUUCCCUGGCUCUAGCAGCA CAGAAAUAUUGGCACAGGGAAGCGAGUCUGCCAAUAUUGGCUGUGCUGCU CCAGGCAGGGUGGUG (SEQ ID NO:7) and mature sequence UAGCAGCACAGAAAUAUUGGC (SEQ ID NO:8) and family consensus sequence UAGCAGCACAGAAAUAUUGGC(-/A) (SEQ ID NO:9)).

let-7a—stem loop UGGGAUGAGGUAGUAGGUUGUA UAGUUUUAGGGUCACACCCACCACUGGGAGAUAACUAUACAAUCUACUGU CUUUCCUA (SEQ ID NO:10) and mature sequence UGAGGUAGUAGGUUGUAUAGUU (SEQ ID NO:11) and family consensus sequence (U/A)GAGGUAGU(A/U)(G/A)(G/U/A)UUG(U/C/A)(A/G/U)U(A/U/G)GU(U/-) (SEQ ID NO:12).

miR-16—stem loop GUCAGCAGUGCCUUAGCAGCACGU AAAUAUUGGCGUUAAGAUUCUAAAAUUAUCUCCAGUAUUAACUGUGCUGC UGAAGUAAGGUUGAC (SEQ ID NO:13) and mature sequence UAGCAGCACGUAAAUAUUGGCG (SEQ ID NO:14) and family consensus sequence UAGCAGCA(C/U)GUAAAUAUUGG(C/G/U/A)(G/U-) (SEQ ID NO:15).

miR-17-5p (miR-17)—stem loop GUCAGAAUAAUGUCAAAGUGCUUACAGU GCAGGUAGUGAUAUGUGCAUCUACUGCAGUGAAGGCACUUGUAGCAUUAU GGUGAC (SEQ ID NO:16) and mature sequence CAAAGUGCUUACAGUGCAGGUAG (SEQ ID NO:17) and family consensus sequence CAAAGUGCUUACAGUGCAGGUA(G/-)(-/U) (SEQ ID NO:18).

miR-24—stem loop CUCCGGUGCCUACUGAGCUGAUAUCAG UUCUCAUUUUACACACUGGCUCAGUUCAGCAGGAACAGGAG (SEQ ID NO:19) and mature sequence UGGCUCAGUUCAGCAGGAACAG (SEQ ID NO:20) and family consensus sequence UGGCUCAGUUCAGCAG(G/-)(A/-)(A/-)(C/-)(A/-)(G/-) (SEQ ID NO:21).

miR-27a—stem loop CUGAGGAGCAGGGCUUAGCUGCUUGUG AGCAGGGUCCACACCAAGUCGUGUUCACAGUGGCUAAGUUCCGCCCCCCAG (SEQ ID NO:22) and mature sequence UUCACAGUGGCUAAGUUCCGC (SEQ ID NO:23) and family consensus sequence UUCACAGUGG(C/U)UAAGUUC(C/A/U)(G/A/U)(C/U/-)(C/U/G/A/-) (SEQ ID NO:24).

miR-30d—stem loop GUUGUUGUAAACAUCCCCGACUGGAAGCUGU AAGACACAGCUAAGCUUUCAGUCAGAUGUUUGCUGCUAC (SEQ ID NO:25) and mature sequence UGUAAACAUCCCCGACUGGAAG (SEQ ID NO:26) and family consensus sequence UGUAAACAU(C/U)CCCGACUGGAAG(-/C)(-/U) (SEQ ID NO:27).

miR-103—stem loop UACUGCCCUCGGCUUCUUUACAGUGCUGCC UUGUUGCAUAUGGAUCAAGCAGCAUUGUACAGGGCUAUGAAGGCAUUG (SEQ ID NO:28) and mature sequence AGCAGCAUUGUACAGGGCUAUGA (SEQ ID NO:29) and family consensus sequence AGCAGCAUUGUACAGGGCUAU(G/C)(A/-) (SEQ ID NO:30).

miR-106a—stem loop CCUUGGCCAUGUAAAAGUGCUUACAGUGCAG GUAGCUUUUUGAGAUCUACUGCAAUGUAAGCACUUCUUACAUUACCAUGG (SEQ ID NO:31) and mature sequence AAAAGUGCUUACAGUGCAGGUAG (SEQ ID NO:32) and family consensus sequence AAAAGUGCUUACAGUGCAGGUA(G/-)(-/C) (SEQ ID NO:33).

miR-125a—stem loop UGCCAGUCUCUAGGUCCCUGAGACCCUUU AACCUGUGAGGACAUCCAGGGUCACAGGUGAGGUUCUUGGGAGCCUGGCG UCUGGCC (SEQ ID NO:34) and mature sequence UCCCUGAGACCCUUUAACCUGUGA (SEQ ID NO:35) and family consensus sequence UCCCUGAGACCCUU(U/-)AACCUGUG(A/-) (SEQ ID NO:36).

miR-143—stem loop GCGCAGCGCCCUGUCUCCCAGCCUGAGG UGCAGUGCUGCAUCUCUGGUCAGUUGGGAGUCUGAGAUGAAGCACUGUAG CUCAGGAAGAGAGAAGUUGUUCUGCAGC (SEQ ID NO:37) and mature sequence UGAGAUGAAGCACUGUAGCUC (SEQ ID NO:38) and family consensus sequence UGAGAUGAAGCACUGUAGCUC(-/G/A) (SEQ ID NO:39).

miR-146a—stem loop CCGAUGUGUAUCCUCAGCUUUGAGAACUGAA UUCCAUGGGUUGUGUCAGUGUCAGACCUCUGAAAUUCAGUUCUUCAGCUG GGAUAUCUCUGUCAUCGU (SEQ ID NO:40) and mature sequence UGAGAACUGAAUUCCAUGGGUU (SEQ ID NO:41) and family consensus sequence UGAGAACUGAAUUCCA(U/A)(G/A)G(G/A)(U/C)(U/G)(-/G) (SEQ ID NO:42).

miR-191—stem loop CGGCUGGACAGCGGGCAACGGAAUCCCAA AAGCAGCUGUUGUCUCCAGAGCAUUCCAGCUGCGCUUGGAUUUCGUCCCCU GCUCUCCUGCCU (SEQ ID NO:43) and mature sequence CAACGGAAUCCCAAAAGCAGCUG (SEQ ID NO:44) and family consensus sequence CAACGGAAUCCCAAAAGCAGCU(G/-) (SEQ ID NO:45).

Corresponding miRNA sequences that can be used in the context of the invention include, but are not limited to, all or a portion of those sequences in SEQ ID NOs: 1-45, as well as the miRNA precursor sequence, or complement of one or more of SEQ ID NOs: 1-45. In some embodiments, an miRNA sequence is or is derived from or contains all or part of miR-221, miR-93, miR-195 to target a particular miRNA (or set of miRNAs) and/or mRNA (or set of mRNAs) for evaluation or modulation.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more miRNA. Consequently, in some embodiments, methods include a step of generating a miRNA profile for a sample. The term “miRNA profile” refers to data regarding the expression pattern of miRNAs in the sample (e.g., one or more of miR-221, miR-93, miR-195, let-7a, miR-16, miR-17-5p, miR-24, miR-27a, miR-30d, miR-103, miR-106a, miR-125a, miR-143, miR-146a, and/or miR-191). It is contemplated that the miRNA profile can be obtained using a set of miRNAs, using for example nucleic acid amplification or hybridization techniques well know to one of ordinary skill in the art. In certain embodiments, expression of one or more of miR-221, miR-93, or miR-195 is evaluated.

In some embodiments of the invention, an miRNA profile is generated by steps that include one or more of: (a) labeling miRNA in the sample; (b) hybridizing miRNA to a number of probes, or amplifying a number of miRNA, and/or (c) determining miRNA hybridization to the probes or detecting miRNA amplification products, wherein a miRNA expression is evaluated. See U.S. Provisional Patent Applications 60/575,743 and 60/649,584, and U.S. patent application Ser. Nos. 11/141,707 and 11/855,792, all of which are hereby incorporated by reference.

Methods of the invention include diagnosing a patient based on miRNA expression. In certain embodiments, the elevation or reduction in the level of expression of a particular miRNA or set of miRNA in a cell is correlated with a disease state as compared to the expression level of that miRNA or set of miRNA in a normal cell or a reference. This correlation allows for diagnostic methods to be carried out when the expression level of a miRNA is measured in a biological sample being assessed and then compared to the expression level of a normal cell or a reference. It is specifically contemplated that miRNA profiles for patients, particularly those suspected of having a particular disease or condition such as lung cancer, can be generated by evaluating any miR or set of miRs discussed in this application. The miRNA profile that is generated from the patient will be one that provides information regarding the particular disease or condition. In many embodiments, the miRNA profile is generated using miRNA hybridization or amplification, (e.g., array hybridization or RT-PCR). In certain aspects, a party evaluating miR expression may prepare a recommendation, report and/or summary conveying processed or raw data to a diagnosing physician. In certain aspects, a miRNA profile can be used in conjunction with other diagnostic tests.

Embodiments of the invention include methods for diagnosing and/or assessing a condition in a patient comprising evaluating expression of one or more miRNAs in a sample from the patient. The difference in the expression in the sample from the patient and a reference, such as expression in a normal or non-pathologic sample, is indicative of a pathologic, disease, or cancerous condition. An miRNA, amplification product, or probe set can comprise a segment of or be complementary to a corresponding miRNA including all or part of miR-221, miR-93, miR-195, let-7a, miR-16, miR-17-5p, miR-24, miR-27a, miR-30d, miR-103, miR-106a, miR-125a, miR-143, miR-146a, and/or miR-191. In certain aspects of the invention, a segment can comprise 1, 2, 3, 4, 5, 6, 7, 8, 9, 10 or more nucleic acid sequence of a miRNA. Other amplification or hybridization sequences may also be included for normalization purposes. The use of an miRNA quantification assay as a clinically relevant diagnostic tool can be enhanced by using an appropriate normalization control. The methods of normalization correct for sample-to-sample variability by comparing a target measurement in a sample to one or more internal controls. Normalization of miRNA quantification assays reduces systematic (non-biological) and non-systematic differences between samples, and can enhance the accurate measurement of differential miRNA expression, for example. The accurate measurement of biologically hardwired differential expression between two groups of samples is the goal of many miRNA qRT-PCR assays. Yet, miRNA levels in qRT-PCR reactions can vary from one sample to the next for reasons that may be technical or biological. Technical reasons may include variabilities in tissue procurement or storage, inconsistencies in RNA extraction or quantification, or differences in the efficiency of the reverse transcription and/or PCR steps. Biological reasons may include sample-to-sample heterogeneity in cellular populations, differences in bulk transcriptional activity, or alterations in specific miRNA expression that is linked to an aberrant biological program (e.g., a disease state). Given the multiplicity of sources that can contribute to differences in miRNA quantification, results from qRT-PCR assays can be normalized against a relevant endogenous target or targets to minimize controllable variation, and permit definitive interpretations of nominal differences in miRNA expression.

With no intent of limiting the invention to any particular theory, most cancers are initially recognized either because signs or symptoms appear, or are identified through screening. Neither of these lead to a definitive diagnosis, which usually requires the opinion of a pathologist. Typically, people with suspected cancer are investigated with various medical tests. These commonly include blood tests, X-rays, CT scans and endoscopy.

A cancer may be suspected for a variety of reasons, but diagnosis of most malignancies is typically confirmed by histological examination of the cancerous cells by a pathologist. Tissue can be obtained from a biopsy or surgery. Many biopsies (such as those of the skin, breast or liver) can be done in a doctor's office. Biopsies of other organs are performed under anesthesia and may require surgery in an operating room.

The tissue diagnosis indicates the type of cell that is proliferating, its histological grade and other features of the tumor. Together, this information is useful to evaluate the prognosis of this patient and choose the best treatment. Cytogenetics and immunohistochemistry may provide information about future behavior of the cancer (prognosis) and best treatment.

A physician may choose to treat a cancer by surgery, chemotherapy, radiation therapy, immunotherapy, monoclonal antibody therapy and/or other methods. The choice of therapy depends upon the location and grade of the tumor and the stage of the disease, as well as the general state of the patient.

Because “cancer” refers to a class of diseases, it is unlikely that there will be a single treatment and aspects of the invention can be used to determine which treatment will be most effective or most harmful and provide a guide for the physician in evaluating, assessing and formulating a treatment strategy for a patient.

In certain aspects, the miR being evaluated is one or more of miR-221, miR-93, or miR-195.

In certain aspects, an increase in expression of one or more miRNA in a patient sample is indicative of lung cancer, wherein the miRNA is one or more of miR-221, miR-93, miR-17-5p, miR-24, miR-27a, miR-103, miR-106a, and/or miR-125a. In certain aspects, the miR is one or more of miR-221 or miR-93.

In further aspects, a decrease in expression of one or more miRNA in a patient sample is indicative of lung cancer, wherein the miRNA is miR-195, miR-16, miR-30d, let-7a, miR-143, miR-146a, and/or miR-191. In certain aspects the miR is miR-195.

A sample may be taken from a patient having or suspected of having a disease or pathological condition. In certain aspects, the sample can be, but is not limited to tissue (e.g., biopsy, particularly fine needle biopsy), sputum, lavage fluid, blood, serum, plasma, lymph node or other tissue or fluid that may contain a lung cancer cell. The sample can be fresh, frozen, fixed (e.g., formalin fixed), or embedded (e.g., paraffin embedded). In a particular aspect, the sample can be a lung sample.

Methods of the invention can be used to diagnose or assess a pathological condition. In certain aspects the condition is a cancerous condition, such as lung cancer and particularly non-small cell lung cancer, small cell lung cancer, squamous cell carcinoma, adenocarcinoma, large cell carcinoma, adenosquamous carcinoma, or undifferentiated carcinoma.

Certain embodiments of the invention include determining expression of one or more miRNA by using an amplification assay or a hybridization assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays.

The methods can further comprise or exclude one or more of steps including: (a) obtaining a sample from the patient, (b) isolating or obtaining nucleic acids from the sample, (c) reverse transcribing nucleic acids from the sample, (d) labeling the nucleic acids isolated from the sample or an amplification product thereof, and/or (e) hybridizing the labeled nucleic acids to one or more probes or detecting the amplified nucleic acids. Nucleic acids of the invention may include one or more nucleic acid comprising at least one segment having a sequence or complementary sequence of one or more of miR-221, miR-93, miR-195, let-7a, miR-16, miR-17-5p, miR-24, miR-27a, miR-30d, miR-103, miR-106a, miR-125a, miR-143, miR-146a, and/or miR-191. In certain aspects, the nucleic acids identify one or more miR-221, miR-93, miR-195, let-7a, miR-16, miR-17-5p, miR-24, miR-27a, miR-30d, miR-103, miR-106a, miR-125a, miR-143, miR-146a, and/or miR-191. In certain aspects, the miR is one or more of miR-221, miR-93, or miR-195. Nucleic acids of the invention may be coupled to a support. Such supports are well known to those of ordinary skill in the art and include, but are not limited to glass, plastic, metal, or latex. In particular aspects of the invention, the support can be planar or in the form of a bead or other geometric shapes or configurations.

Aspects of the invention can be used to diagnose or assess a patient's condition. For example, the methods can be used to screen for a pathological condition, assess prognosis of a pathological condition, stage a pathological condition, or assess response of a pathological condition to therapy.

Other embodiments of the invention concern nucleic acids that perform the activities of or inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA. In certain aspects, short nucleic acid molecules that function as miRNAs or as inhibitors of miRNA in a cell. The term “short” refers to a length of a single polynucleotide that is 10, 15, 20, 25, 50, 100, or 150 nucleotides or fewer, including all integers or range derivable there between.

The introduction of a nucleic acid can result in reducing or eliminating activity of one or more miRNAs in a cell comprising introducing into a cell a miRNA inhibitor; or supplying or enhancing the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or a miRNA inhibitor are synthetic.

Methods include identifying a cell or patient in need of inducing those cellular characteristics. Identification of such a cell may be, for example, by evaluating the expression of one or more of miR-221, miR-93, miR-195, let-7a, miR-16, miR-17-5p, miR-24, miR-27a, miR-30d, miR-103, miR-106a, miR-125a, miR-143, miR-146a, and/or miR-191. Also, it will be understood that an amount of a synthetic nucleic acid that is provided to a cell or organism is an “effective amount,” which refers to an amount needed to achieve a desired goal, such as inducing a particular cellular characteristic(s).

The present invention also concerns kits containing compositions of the invention or compositions to implement methods of the invention. In some embodiments, kits can be used to evaluate one or more miRNA molecules. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more miRNA probes, synthetic miRNA molecules or miRNA inhibitors, or any range and combination derivable therein. In some embodiments, there are kits for evaluating miRNA activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using miRNA probes, synthetic miRNAs, nonsynthetic, and/or miRNA inhibitors of the invention for therapeutic, prognostic, or diagnostic applications are included as part of the invention. Specifically contemplated are any such molecules corresponding to any miRNA diagnostic of lung disease, such as those discussed herein.

In certain aspects, negative and/or positive control synthetic miRNAs and/or miRNA inhibitors are included in some kit embodiments. The Control molecules can be used to verify transfection efficiency and/or control for transfection-induced changes in cells.

It is contemplated that any method or composition described herein can be implemented with respect to any other method or composition described herein and that different embodiments may be combined. It is specifically contemplated that any methods and compositions discussed herein with respect to miRNA molecules or miRNA may be implemented with respect to synthetic miRNAs to the extent the synthetic miRNA is exposed to the proper conditions to allow it to become a mature miRNA under physiological circumstances. The claims originally filed are contemplated to cover claims that are multiply dependent on any filed claim or combination of filed claims.

Any embodiment of the invention involving specific miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence of the specified miRNA. In other aspects miRNA of the invention may include additional nucleotides at the 5′, 3′, or both 5′ and 3′ ends of at least, at most or about 1, 2, 3, 4, 5, 6, 7, 8, 9, 10 or more nucleotides.

Embodiments of the invention include kits for analysis of a pathological sample by assessing miRNA profile for a sample comprising, in suitable container means, two or more miRNA probes and/or amplification primers, wherein the miRNA probes detect or primer amplify one or more of miR-221, miR-93, miR-195, let-7a, miR-16, miR-17-5p, miR-24, miR-27a, miR-30d, miR-103, miR-106a, miR-125a, miR-143, miR-146a, and/or miR-191. The kit can further comprise reagents for labeling miRNA in the sample. The kit may also include the labeling reagents include at least one amine-modified nucleotide, poly(A) polymerase, and poly(A) polymerase buffer. Labeling reagents can include an amine-reactive dye.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Any embodiment discussed with respect to a particular lung disorder can be applied or implemented with respect to a different lung disorder. Furthermore, compositions and kits of the invention can be used to achieve methods of the invention.

Throughout this application, the term “about” may be used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DETAILED DESCRIPTION OF THE INVENTION

The present invention is directed to compositions and methods relating to preparation and characterization of miRNAs, as well as use of miRNAs for therapeutic, prognostic, and diagnostic applications, particularly those methods and compositions related to assessing and/or identifying lung disease.

### I. LUNG DISEASE AND CANCER

Lung cancer is the leading cause of cancer death worldwide in both men and women and is primarily caused by tobacco smoking. In 2007, approximately 160,000 people will die of lung cancer in the United States alone (Jemal et al., 2007). Lung cancers are generally divided into two types (non-small cell lung cancers and small cell lung cancers). Non-small cell cancers are more common and include squamous cell carcinoma, adenocarcinoma, large cell carcinoma, adenosquamous carcinoma, and undifferentiated carcinoma. Among these, squamous cell carcinomas and adenocarcinomas are the most common types.

It is important to determine the type of lung cancer, as different carcinoma types involve different regions of the lung and are treated differently. Identification of a specific lung cancer type is usually initiated by an examination of cells in sputum (sputum cytology) followed by examination of cells collected from the lung or bronchus, usually by flexible bronchoscopy or fine needle aspiration (FNA). Once a tissue diagnosis has been established, a thorough staging analysis, including metastatic evaluation and staging classification, is frequently performed.

Lung cancer, or carcinoma of the lung, is a disease where epithelial (internal lining) tissue in the lung grows out of control. This leads to metastasis, invasion of adjacent tissue and infiltration beyond the lungs. Lung cancer, the most common cause of cancer-related death in men and the second most common in women, is responsible for 1.3 million deaths worldwide annually. The most common symptoms are shortness of breath, cough (including coughing up blood), and weight loss.

Lung cancer may be seen on chest x-ray and computed tomography (CT scan). The diagnosis is typically confirmed with a biopsy. This is usually performed via bronchoscopy or CT-guided biopsy.

Treatment and prognosis depend upon the histological type of cancer, the stage (degree of spread), and the patient's performance status. Possible treatments include surgery, chemotherapy, and radiotherapy. With treatment, the five-year survival rate is 14%.

The main types of lung cancer are small cell lung carcinoma and non-small cell lung carcinoma. Non-small cell lung carcinoma (NSCLC) is sometimes treated with surgery, while small cell lung carcinoma (SCLC) usually responds better to chemotherapy.

Non-small cell lung carcinoma (NSCLC)—The non-small cell lung carcinomas are grouped together because their prognosis and management are similar. There are three main sub-types: squamous cell lung carcinoma, adenocarcinoma and large cell lung carcinoma. Sub-types of non-small cell lung cancer include squamous cell lung carcinoma, adenocarcinoma (e.g., bronchioloalveolar carcinoma, adenosquamous carcinoma, papillary adenocarcinoma, mucoepidermoid carcinoma, adenoid cystic carcinoma), large cell carcinoma, and giant cell and spindle cell carcinoma.

Squamous cell lung carcinoma usually starts near a central bronchus. Adenocarcinoma usually originates in peripheral lung tissue. Among people who have never smoked (“never-smokers”), adenocarcinoma is the most common form of lung cancer. Bronchioloalveolar carcinoma, is more common in female never-smokers. Large cell lung carcinoma is a fast-growing form that develops near the surface of the lung. It is often poorly differentiated and tends to metastasize early.

Small cell lung carcinoma (SCLC)—Small cell lung carcinoma (SCLC, also called “oat cell carcinoma”) is less common. It tends to arise in the larger breathing tubes and grows rapidly, becoming quite large. The “oat” cell contains dense neurosecretory granules (vesicles containing neuroendocrine hormones) which give this an endocrine/paraneoplastic syndrome association. While initially more sensitive to chemotherapy, it ultimately carries a worse prognosis and is often metastatic at presentation. This type of lung cancer is strongly associated with smoking. Primary lung cancers themselves most commonly metastasize to the adrenal glands, liver, brain, and bone.

Lung cancer staging is an assessment of the degree of spread of the cancer from its original source. It is an important factor affecting the prognosis and potential treatment of lung cancer.

Non-small cell lung carcinoma is staged from IA (“one A”, best prognosis) to IV (“four”, worst prognosis). Small cell lung carcinoma is classified as limited stage if it is confined to one half of the chest and within the scope of a single radiotherapy field. Otherwise it is extensive stage.

Diagnosis typically includes performing a chest x-ray if a patient reports symptoms that may be suggestive of lung cancer. This may reveal an obvious mass, widening of the mediastinum (suggestive of spread to lymph nodes there), atelectasis (collapse), consolidation (pneumonia), or pleural effusion. If there are no x-ray findings but the suspicion is high (such as a heavy smoker with blood-stained sputum), bronchoscopy and/or a CT scan may provide the necessary information. Bronchoscopy or CT-guided biopsy is often used to identify the tumor type.

The differential diagnosis for patients who present with abnormalities on chest x-ray includes lung cancer, as well as nonmalignant diseases. These include infectious causes such as tuberculosis or pneumonia, or inflammatory conditions such as sarcoidosis. These diseases can result in mediastinal lymphadenopathy or lung nodules, and sometimes mimic lung cancers.

Biomarkers for lung cancer include analysis of mRNA expression. Examples of some recent studies include hnRNPB1, an RNA-binding protein involved in mRNA transportation and RNA mutation that is common in squamous cell carcinoma of the lung. The protein has been found to be overexpressed in early-stage lung cancer.

Human telomeres function as a protective structure capping the ends of chromosomes. Dysfunction plays an important role in cancer initiation and progression. Human telomerase catalytic component (hTERT) is known to be elevated in cancers and epidermal growth factor receptor (EGFR) is often increased in lung cancer. Expression of repressor activator protein 1 (RAP1), a gene that helps regulate telomere stability, was associated with improved survival in lung cancer patients.

Survivin is a protein that inhibits apoptosis and promotes mitosis. The amount of nuclear survivin in tumor tissue may predict recurrence and poor survival in patients with resected NSCLC. Osteopontin overexpression has been found in both tissue and serum in lung cancer patients, and may be associated with more aggressive disease. Other mRNA that may be included in such an analysis includes, but is not limited to CYFRA 21.1 (Molina et al., 1994) and/or carcino embryonic antigen (CEA) (Aznar et al. 1997).

Analysis of mRNA expression and/or standard diagnostic assays can be used in conjunction with evaluating the levels of miRNA to diagnose or differentiate lung cancer.

### II. EVALUATION OF MIRNA LEVELS

It is contemplated that a number of assays could be employed to analyze miRNAs, their activities, and their effects. Such assays include, but are not limited to, array hybridization, solution hybridization, nucleic amplification, polymerase chain reaction, quantitative PCR, RT-PCR, in situ hybridization, Northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Oligo Ligation Assay (OLA), hybridization, and array analysis.

U.S. patent application Ser. Nos. 11/141,707, filed May 31, 2005; 11/857,948, filed Sep. 19, 2007; 11/273,640, filed Nov. 14, 2005 and provisional patent application 60/869,295, filed Dec. 8, 2006 are incorporated by reference in their entirety.

A. Sample Preparation

While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from lavage, biopsy, fine needle aspirates, exfoliates, blood, sputum, tissue, organs, semen, saliva, tears, urine, cerebrospinal fluid, body fluids, hair follicles, skin, or any sample containing or constituting biological cells. In certain embodiments, samples may be, but are not limited to, fresh, frozen, fixed, formalin fixed, preserved, RNA later preserved, paraffin embedded, or formalin fixed and paraffin embedded. Alternatively, the sample may not be a biological sample, but be a chemical mixture, such as a cell-free reaction mixture (which may contain one or more biological enzymes).

B. Differential Expression Analyses

Methods of the invention can be used to detect differences in miRNA expression or levels between two samples, or a sample and a reference (e.g., a tissue reference or a digital reference representative of a non-cancerous state). Specifically contemplated applications include identifying and/or quantifying differences between miRNA from a sample that is normal and from a sample that is not normal, between a cancerous condition and a non-cancerous condition, or between two differently treated samples (e.g., a pretreatment versus a posttreatment sample). Also, miRNA may be compared between a sample believed to be susceptible to a particular therapy, disease, or condition and one believed to be not susceptible or resistant to that therapy, disease, or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of a disease or condition of which a component is or may or may not be genetic or caused by a hyperproliferative or neoplastic cell or cells.

It is specifically contemplated that the invention can be used to evaluate differences between stages of disease, such as between hyperplasia, neoplasia, pre-cancer and cancer, or between a primary tumor and a metastasized tumor.

Phenotypic traits also include characteristics such as longevity, morbidity, susceptibility or receptivity to particular drugs or therapeutic treatments (drug efficacy), and risk of drug toxicity.

In certain embodiments, miRNA profiles may be generated to evaluate and correlate those profiles with pharmacokinetics. For example, miRNA profiles may be created and evaluated for patient tumor and blood samples prior to the patient's being treated or during treatment to determine if there are miRNAs whose expression correlates with the outcome of treatment. Identification of differential miRNAs can lead to a diagnostic assay involving them that can be used to evaluate tumor and/or blood samples to determine what drug regimen the patient should be provided. In addition, it can be used to identify or select patients suitable for a particular clinical trial. If a miRNA profile is determined to be correlated with drug efficacy or drug toxicity that may be relevant to whether that patient is an appropriate patient for receiving the drug or for a particular dosage of the drug.

In addition to the above assay, blood samples from patients can be evaluated to identify a disease or a condition based on miRNA levels, such as metastatic disease. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005, which is hereby incorporated by reference in its entirety.

C. Amplification

Many methods exist for evaluating miRNA levels by amplifying all or part of miRNA nucleic acid sequences such as mature miRNAs, precursor miRNAs, and primary miRNAs. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain embodiments, more than one amplification method may be used, such as reverse transcription followed by real time PCR (Chen et al., 2005 and/or U.S. patent application Ser. No. 11/567,082, filed Dec. 5, 2006, which are incorporated herein by reference in its entirety).

A typical PCR reaction includes multiple amplification steps, or cycles that selectively amplify target nucleic acid species. A typical PCR reaction includes three steps: a denaturing step in which a target nucleic acid is denatured; an annealing step in which a set of PCR primers (forward and reverse primers) anneal to complementary DNA strands; and an elongation step in which a thermostable DNA polymerase elongates the primers. By repeating these steps multiple times, a DNA fragment is amplified to produce an amplicon, corresponding to the target DNA sequence. Typical PCR reactions include 20 or more cycles of denaturation, annealing, and elongation. In many cases, the annealing and elongation steps can be performed concurrently, in which case the cycle contains only two steps. Since mature miRNAs are single stranded, a reverse transcription reaction (which produces a complementary cDNA sequence) is performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than 15, 20, 25, 30, or 35 nucleotides in length. In additional embodiments, a primer is at least 35 nucleotides in length.

In a further aspect, a forward primer can comprise at least one sequence that anneals to a target miRNA and alternatively can comprise an additional 5′ noncomplementary region. In another aspect, a reverse primer can be designed to anneal to the complement of a reverse transcribed miRNA. The reverse primer may be independent of the miRNA sequence, and multiple miRNAs may be amplified using the same reverse primer. Alternatively, a reverse primer may be specific for a miRNA.

In some embodiments, two or more miRNAs or nucleic acids are amplified in a single reaction volume or multiple reaction volumes. In certain aspects, one or more miRNA or nucleic may be used as a normalization control or a reference nucleic acid for normalization. Normalization may be performed in separate or the same reaction volumes as other amplification reactions. One aspect includes multiplex q-PCR, such as qRT-PCR, which enables simultaneous amplification and quantification of at least one miRNA of interest and at least one reference nucleic acid in one reaction volume by using more than one pair of primers and/or more than one probe. The primer pairs comprise at least one amplification primer that uniquely binds each nucleic acid, and the probes are labeled such that they are distinguishable from one another, thus allowing simultaneous quantification of multiple miRNAs. Multiplex qRT-PCR has research and diagnostic uses, including but not limited to detection of miRNAs for diagnostic, prognostic, and therapeutic applications.

A single combined reaction for q-PCR, may be used to: (1) decreased risk of experimenter error, (2) reduction in assay-to-assay variability, (3) decreased risk of target or product contamination, and (4) increased assay speed. The qRT-PCR reaction may further be combined with the reverse transcription reaction by including both a reverse transcriptase and a DNA-based thermostable DNA polymerase. When two polymerases are used, a “hot start” approach may be used to maximize assay performance (U.S. Pat. Nos. 5,411,876 and 5,985,619). For example, the components for a reverse transcriptase reaction and a PCR reaction may be sequestered using one or more thermoactivation methods or chemical alteration to improve polymerization efficiency (U.S. Pat. Nos. 5,550,044, 5,413,924, and 6,403,341).

To assess the expression of microRNAs, real-time RT-PCR detection can be used to screen nucleic acids or RNA isolated from samples of interest and a related reference such as normal adjacent tissue (NAT) samples.

A panel of amplification targets is chosen for real-time RT-PCR quantification. The selection of the panel or targets can be based on the results of microarray expression analyses, such as mirVana™ miRNA Bioarray V1, Ambion. In one aspect, the panel of targets includes one or more of hsa-let-7a, hsa-miR-16, hsa-miR-17-5p, hsa-miR-24, hsa-miR-27a, hsa-miR-30d, hsa-miR-93, hsa-miR-103, hsa-miR-106a, hsa-miR-125a, hsa-miR-143, hsa-miR-146a, hsa-miR-191, hsa-miR-195, and hsa-miR-221. One example of a normalization target is 5S rRNA and other can be included. Reverse transcription (RT) reaction components are typically assembled on ice prior to the addition of RNA template. Total RNA template is added and mixed. RT reactions are incubated in an appropriate PCR System at an appropriate temperature (15-30° C., including all values and ranges there between) for an appropriate time, 15 to 30 minutes or longer, then at a temperature of 35 to 42 to 50° C. for 10 to 30 to 60 minutes, and then at 80 to 85 to 95° C. for 5 minutes, then placed on wet ice. Reverse Transcription reaction components typically include nuclease-free water, reverse transcription buffer, dNTP mix, RT Primer, RNase Inhibitor, Reverse Transcriptase, and RNA.

PCR reaction components are typically assembled on ice prior to the addition of the cDNA from the RT reactions. Following assembly of the PCR reaction components a portion of the RT reaction is transferred to the PCR mix. PCR reaction are then typically incubated in an PCR system at an elevated temperature (e.g., 95° C.) for 1 minute or so, then for a number of cycles of denaturing, annealing, and extension (e.g., 40 cycles of 95° C. for 5 seconds and 60° C. for 30 seconds). Results can be analyzed, for example, with SDS V2.3 (Applied Biosystems). Real-time PCR components typically include Nuclease-free water, MgCl₂, PCR Buffer, dNTP mix, one or more primers, DNA Polymerase, cDNA from RT reaction and one or more detectable label.

Software tools such as NormFinder (Andersen et al., 2004) are used to determine targets for normalization with the targets of interest and tissue sample set. For normalization of the real-time RT-PCR results, the cycle threshold (Cₜ) value (a log value) for the microRNA of interest is subtracted from the geometric mean Cₜ value of normalization targets. Fold change can be determined by subtracting the dCₜ normal reference (N) from the corresponding dCₜ sample being evaluated (T), producing a ddCₜ(T−N) value for each sample. The average ddCₜ(T−N) value across all samples is converted to fold change by 2^{ddCt}. The representative p-values are determined by a two-tailed paired Student's t-test from the dCₜ values of sample and normal reference.

D. Nucleic Acid Arrays

Certain aspects of the present invention concerns the preparation and use of miRNA arrays or miRNA probe arrays, which are ordered macroarrays or microarrays of nucleic acid molecules (probes) that are fully or nearly complementary or identical to a plurality of miRNA molecules or precursor miRNA molecules and are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference. Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

E. Hybridization

After an array or a set of miRNA probes is prepared and the miRNA in the sample is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

### III. RNA MOLECULES

MicroRNA (“miRNA” or “miR”) molecules are generally 21 to 22 nucleotides in length, though lengths of 17 and up to 24 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) become part of a large complex to down-regulate a particular target gene. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

It is understood that a “synthetic nucleic acid” of the invention means that the nucleic acid does not have a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

It will be understood that the term “naturally occurring” refers to something found in an organism without any intervention by a person; it could refer to a naturally-occurring wildtype or mutant molecule.

The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together.

In certain aspects, synthetic miRNA of the invention are RNA or RNA analogs. miRNA inhibitors may be DNA or RNA, or analogs thereof. Nucleic acid based miRNA and miRNA inhibitors of the invention are collectively referred to as “synthetic nucleic acids.” In other aspects, an miRNA inhibitor can be a protein or a polypeptide that interacts with an endogenous miRNA or processing.

In some embodiments, there is a synthetic or isolated miRNA having a length of between 17 and 130 residues. The present invention concerns synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range derivable therein.

In certain embodiments, synthetic miRNA have (a) a “miRNA region” whose sequence from 5′ to 3′ is identical to all or a segment of a mature miRNA sequence, and (b) a “complementary region” whose sequence from 5′ to 3′ is between 60% and 100% complementary to the miRNA sequence. In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA. Alternatively, the miRNA region can comprise 18, 19, 20, 21, 22, 23, 24 or more nucleotide positions in common with a naturally-occurring miRNA as compared by sequence alignment algorithms and methods well known in the art.

The term “complementary region” refers to a region of a synthetic miRNA that is or is at least 60% complementary to the mature, naturally occurring miRNA sequence that the miRNA region is identical to. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, a miRNA inhibitor has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the probe sequence that is complementary to the sequence of a mature miRNA as the sequence for a miRNA inhibitor. Moreover, that portion of the probe sequence can be altered so that it is still 90% complementary to the sequence of a mature miRNA.

In some embodiments, of the invention, a synthetic miRNA contains one or more design elements. These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluoroscein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there is one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification. In further embodiments, there is one or more sugar modifications in the first or last 2 to 4 residues of the complementary region or the first or last 4 to 6 residues of the complementary region. This design element can also be used with a miRNA inhibitor. Thus, a miRNA inhibitor can have this design element and/or a replacement group on the nucleotide at the 5′ terminus, as discussed above.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there is a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

In some embodiments of the invention, methods and compositions involving miRNA may concern miRNA and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, control nucleic acids, and other probes and primers. In many embodiments, miRNA are 19-24 nucleotides in length, while miRNA probes are 5, 10, 15, 19, 20, 25, 30, to 35 nucleotides in length, including all values and ranges there between, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or 110 contiguous nucleotides. It is further understood that the length of complementarity within a precursor miRNA or between a miRNA probe and a miRNA or a miRNA gene are such lengths. Moreover, the complementarity may be expressed as a percentage, meaning that the complementarity between a probe and its target is 90% identical or greater over the length of the probe. In some embodiments, complementarity is or is at least 90%, 95% or 100% identical. In particular, such lengths may be applied to any nucleic acid comprising a nucleic acid sequence identified in any of SEQ ID NO:1 through SEQ ID NO:45 or any other sequence disclosed herein.

The term “recombinant” may be used and this generally refers to a molecule that has been manipulated in vitro or that is a replicated or expressed product of such a molecule.

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

Nucleic acids of the invention may be made by any technique known to one of ordinary skill in the art, such as for example, chemical synthesis, enzymatic production or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

A. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for form a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried down and resuspended in a liquid and volume appropriate for subsequent manipulation.

B. Preparation of Nucleic Acids

Alternatively, nucleic acid synthesis is performed according to standard methods. See, for example, Itakura and Riggs (1980). Additionally, U.S. Pat. Nos. 4,704,362, 5,221,619, and 5,583,013 each describe various methods of preparing synthetic nucleic acids. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

In certain embodiments, the present invention concerns nucleic acid molecules that are not synthetic. In some embodiments, the nucleic acid molecule has a chemical structure of a naturally occurring nucleic acid and a sequence of a naturally occurring nucleic acid, such as the exact and entire sequence of a single stranded primary miRNA (see Lee 2002), a single-stranded precursor miRNA, or a single-stranded mature miRNA. In addition to the use of recombinant technology, such non-synthetic nucleic acids may be generated chemically, such as by employing technology used for creating oligonucleotides.

C. Labels and Labeling Techniques

In some embodiments, the present invention concerns miRNA that are directly or indirectly labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. The source of the enzyme is not limiting. Examples of sources for the enzymes include yeast, gram-negative bacteria such as E. coli, lactococcus lactis, and sheep pox virus.

Enzymes capable of adding such nucleotides include, but are not limited to, poly(A) polymerase, terminal transferase, and polynucleotide phosphorylase. In specific embodiments of the invention, a ligase is contemplated as not being the enzyme used to add the label, and instead, a non-ligase enzyme is employed.

Terminal transferase catalyzes the addition of nucleotides to the 3′ terminus of a nucleic acid.

Polynucleotide phosphorylase can polymerize nucleotide diphosphates without the need for a primer.

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phycoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′, 4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### IV. THERAPEUTIC METHODS

Certain embodiments of the invention concern nucleic acids that perform the activities of or inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA.

The present invention concerns, in some embodiments, short nucleic acid molecules that function as miRNAs or as inhibitors of miRNA in a cell. The term “short” refers to a length of a single polynucleotide that is 15, 16, 17, 18, 19, 20, 21, 22, 23, 24, 25, 50, 100, or 150 nucleotides or fewer, including all integers or ranges derivable there between. The nucleic acid molecules are typically synthetic. The term “synthetic” refers to nucleic acid molecule that is isolated and not produced naturally in a cell. In certain aspects the sequence (the entire sequence) and/or chemical structure deviates from a naturally-occurring nucleic acid molecule, such as an endogenous precursor miRNA or miRNA molecule or complement thereof.

Generally, inhibitors of miRNAs can be given to achieve the opposite effect as compared to when nucleic acid molecules corresponding to the mature miRNA are given. Similarly, nucleic acid molecules corresponding to the mature miRNA can be given to achieve the opposite effect as compared to when inhibitors of the miRNA are given. For example, miRNA molecules that increase cell proliferation can be provided to cells to increase proliferation or inhibitors of such molecules can be provided to cells to decrease cell proliferation. The present invention contemplates these embodiments in the context of the different physiological effects observed with the different miRNA molecules and miRNA inhibitors disclosed herein. These include, but are not limited to, the following physiological effects: increase and decreasing cell proliferation, increasing or decreasing apoptosis, increasing transformation, increasing or decreasing cell viability, activating ERK, activating/inducing or inhibiting hTert, inhibit stimulation of Stat3, reduce or increase viable cell number, and increase or decrease number of cells at a particular phase of the cell cycle.

Methods of the invention are generally contemplated to include providing or introducing one or more different nucleic acid molecules corresponding to one or more different miRNA molecules. It is contemplated that the following, at least the following, or at most the following number of different nucleic acid molecules may be provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or any range derivable therein. This also applies to the number of different miRNA molecules that can be provided or introduced into a cell.

Furthermore, it is contemplated that the miRNA compositions may be provided as part of a therapy to a patient, in conjunction with traditional therapies or preventative agents. Moreover, it is contemplated that any method discussed in the context of therapy may be applied as preventatively, particularly in a patient identified to be potentially in need of the therapy or at risk of the condition or disease for which a therapy is needed.

In addition, methods of the invention concern employing one or more nucleic acids corresponding to a miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating cancer in a patient comprising administering to the patient the cancer therapeutic and an effective amount of at least one miRNA molecule that improves the efficacy of the cancer therapeutic or protects non-cancer cells.

Cancer therapies also include a variety of combination therapies with both chemical and radiation based treatments. Combination chemotherapies include but are not limited to, for example, 5-fluorouracil, alemtuzumab, amrubicin, bevacizumab, bleomycin, bortezomib, busulfan, camptothecin, capecitabine, cisplatin (CDDP), carboplatin, cetuximab, chlorambucil, cisplatin (CDDP), cyclophosphamide, camptothecin, COX-2 inhibitors (e.g., celecoxib), cyclophosphamide, cytarabine, dactinomycin, dasatinib, daunorubicin, dexamethasone, docetaxel, doxorubicin (adriamycin), EGFR inhibitors (gefitinib and cetuximab), erlotinib, estrogen receptor binding agents, etoposide (VP16), everolimus, farnesyl-protein transferase inhibitors, gefitinib, gemcitabine, gemtuzumab, ibritumomab, ifosfamide, imatinib mesylate, larotaxel, lapatinib, lonafarnib, mechlorethamine, melphalan, methotrexate, mitomycin, navelbine, nitrosurea, nocodazole, oxaliplatin, paclitaxel, plicomycin, procarbazine, raloxifene, rituximab, sirolimus, sorafenib, sunitinib, tamoxifen, taxol, taxotere, temsirolimus, tipifarnib, tositumomab, transplatinum, trastuzumab, vinblastin, vincristin, or vinorelbine or any analog or derivative variant of the foregoing.

Methods of the present invention include the delivery of an effective amount of a miRNA or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease.

A. Administration

In certain embodiments, it is desired to kill cells, inhibit cell growth, inhibit metastasis, decrease tumor or tissue size, and/or reverse or reduce the malignant or disease phenotype of cells. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation. Direct injection, intratumoral injection, or injection into tumor vasculature is specifically contemplated for discrete, solid, accessible tumors, or other accessible target areas. Local, regional, or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as a single dose comprise about 0.1 to about 0.5 ml volumes. Compositions of the invention may be administered in multiple injections to a tumor or a targeted site. In certain aspects, injections may be spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a miRNA or combinations thereof. Administration may be continued post-resection, for example, by leaving a catheter implanted at the site of the surgery. Periodic post-surgical treatment also is envisioned. Continuous perfusion of an expression construct or a viral construct also is contemplated.

Treatment regimens may vary as well and often depend on tumor type, tumor location, immune condition, target site, disease progression, and health and age of the patient. Certain tumor types will require more aggressive treatment. The clinician will be best suited to make such decisions based on the known efficacy and toxicity (if any) of the therapeutic formulations.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. The amount specified may be the amount administered as the average daily, average weekly, or average monthly dose. miRNA can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μg or mg, or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

B. Injectable Compositions and Formulations

In some embodiments, the method for the delivery of a miRNA or an expression construct encoding such or combinations thereof is via systemic administration. However, the pharmaceutical compositions disclosed herein may also be administered parenterally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

In certain formulations, a water-based formulation is employed while in others, it may be lipid-based. In particular embodiments of the invention, a composition comprising a tumor suppressor protein or a nucleic acid encoding the same is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral, intralesional, and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The phrase “pharmaceutically acceptable” refers to molecular entities and compositions that do not produce an allergic or similar untoward reaction when administered to a human.

The nucleic acid(s) are administered in a manner compatible with the dosage formulation, and in such amount as will be therapeutically effective. The quantity to be administered depends on the subject to be treated, including, e.g., the aggressiveness of the disease or cancer, the size of any tumor(s) or lesions, the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

C. Combination Treatments

In certain embodiments, the compositions and methods of the present invention involve a miRNA, or expression construct encoding such. These miRNA compositions can be used in combination with a second therapy to enhance the effect of the miRNA therapy, or increase the therapeutic effect of another therapy being employed. These compositions would be provided in a combined amount effective to achieve the desired effect, such as the killing of a cancer cell and/or the inhibition of cellular hyperproliferation. This process may involve contacting the cells with the miRNA or second therapy at the same or different time. This may be achieved by contacting the cell with one or more compositions or pharmacological formulation that includes or more of the agents, or by contacting the cell with two or more distinct compositions or formulations, wherein one composition provides (1) miRNA; and/or (2) a second therapy. A second composition or method may be administered that includes a chemotherapy, radiotherapy, surgical therapy, immunotherapy or gene therapy.

It is contemplated that one may provide a patient with the miRNA therapy and the second therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example miRNA therapy is “A” and a second therapy is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a second therapy, such as chemotherapy, radiotherapy, immunotherapy, surgical therapy or other gene therapy, is employed in combination with the miRNA therapy, as described herein.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Alkylating agents include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents.

Antimetabolites include 5-fluorouracil (5-FU), cytarabine (Ara-C), fludarabine, gemcitabine, and methotrexate.

Antitumor antibiotics include bleomycin, dactinomycin, daunorubicin, doxorubicin (Adriamycin), and idarubicin.

Mitotic inhibitors include plant alkaloids and other natural agents that can inhibit either protein synthesis required for cell division or mitosis. Mitotic inhibitors include docetaxel, etoposide (VP16), paclitaxel, taxol, taxotere, vinblastine, vincristine, and vinorelbine.

Nitrosureas include carmustine and lomustine.

2. Radiotherapy

Radiotherapy, also called radiation therapy, is the treatment of cancer and other diseases with ionizing radiation. Radiation therapy used according to the present invention may include, but is not limited to, the use of γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. Radiotherapy may comprise the use of radiolabeled antibodies to deliver doses of radiation directly to the cancer site (radioimmunotherapy). Once injected into the body, the antibodies actively seek out the cancer cells, which are destroyed by the cell-killing (cytotoxic) action of the radiation. This approach can minimize the risk of radiation damage to healthy cells.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment a specially made metal frame is attached to the head. Then, several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor or disease cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). A non-limiting list of several known anti-cancer immunotherapeutic agents include Cetuximab, Panitumumab, Trastuzumab, Bevacizumab, Alemtuzumab, Gemtuzumab ozogamicin, Rituximab, Tositumomab, Matuzumab, Ibritumomab tiuxetan, Tositumomab, HuPAM4, MORAb-009, G250, mAb 8H9, M195, Ipilimumab, HuLuc63, Alemtuzumab, Epratuzumab, BC8, HuJ591, hA20, Lexatumumab, Pertuzumab, Mik-beta-1, RAV12, SGN-30, AME-133v, HeFi-1, BMS-663513, Volociximab, GC1008, HCD122, Siplizumab, MORAb-003, CNTO 328, MDX-060, Ofatumumab, or SGN-33. It is contemplated that one or more of these therapies may be employed with the miRNA therapies described herein.

4. Gene Therapy

In yet another embodiment, a combination treatment involves gene therapy in which a therapeutic polynucleotide is administered before, after, or at the same time as one or more therapeutic miRNA. Delivery of a therapeutic polypeptide or encoding nucleic acid in conjunction with a miRNA may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

This application incorporates U.S. application Ser. No. 11/349,727 filed on Feb. 8, 2006 claiming priority to U.S. Provisional Application Ser. No. 60/650,807 filed Feb. 8, 2005 herein by references in its entirety.

### V. KITS

Any of the compositions or components described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from lung samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, magnetic beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: 1) poly(A) polymerase; 2) unmodified nucleotides (G, A, T, C, and/or U); 3) a modified nucleotide (labeled or unlabeled); 4) poly(A) polymerase buffer; and, 5) at least one microfilter; 6) label that can be attached to a nucleotide; 7) at least one miRNA probe; 8) reaction buffer; 9) a miRNA array or components for making such an array; 10) acetic acid; 11) alcohol; 12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (lung cancer), (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ ID NOS:1-45. In certain embodiments, a kit or array of the invention can contain one or more probes for the miRNAs identified by SEQ ID NOS:1-45. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquotted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Such kits may also include components that facilitate isolation of the labeled miRNA. It may also include components that preserve or maintain the miRNA or that protect against its degradation. Such components may be RNase-free or protect against RNases. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### VI. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art. Unless otherwise designated, catalog numbers refer to products available by that number from Ambion, Inc.®, The RNA Company.

### Example 1

### Real-Time RT-PCR for Evaluation of MicroRNA Expression in Human Lung Tumor Total RNA

To assess the expression of microRNAs, real-time RT-PCR detection with TaqMan® MicroRNA Assays (Applied Biosystems, Foster City, Calif., USA) was used to screen FirstChoice® Total RNA (Ambion, Austin, Tex., USA) isolated from 12 pairs of human lung tumor and normal adjacent tissue (NAT) samples. Details of pathology for each tumor sample are given in Table 1.

TABLE 1
Human Lung Tumor Sample Pathology (tumor sizes reported in cm).

| | | | TNM | |
|---|---|---|---|---|
| Tumor | Patient | Patient | Staging | |
| ID | Age | Sex | Score | Diagnosis |
| 1 | 68 | M | T2 N0 MX | Lung Cancer Squamous cell carcinoma |
| | | | | moderately differentiated |
| 2 | 56 | M | T2 N0 M0 | Lung Cancer Squamous cell carcinoma |
| | | | | moderately differentiated, Stage 1B |
| 3 | 47 | M | T2 N0 M0 | Lung Cancer, Squamous cell carcinoma, |
| | | | | Stage 1B |
| 4 | 67 | M | T2 N0 M0 | Central Lung Cancer, Squamous cell |
| | | | | carcinoma, poorly differentiated, Stage 1B |
| 5 | 66 | M | T2 N1 M0 | Lung Cancer, Squamous cell carcinoma well |
| | | | | differentiated, Stage 2A |
| 6 | 68 | M | T2 N0 MX | Lung Cancer Squamous cell carcinoma |
| | | | | moderately differentiated |
| 7 | 57 | M | T3 N1 M0 | Squamous cell carcinoma, Right lung |
| | | | | pneumonectomy, Stage 3A, tumor size = |
| | | | | 4 × 3.5 × 3.5 |
| 8 | 65 | M | T2 N0 M0 | Squamous cell carcinoma, Right upper lobe |
| | | | | lobectomy, Stage 1B, tumor size = 7 × 6 × 4 |
| 9 | 63 | M | T2 N1 M0 | Squamous cell carcinoma, Right |
| | | | | pneumonectomy, Stage 2B, tumor size = |
| | | | | 6.8 × 6.5 × 6.0 |
| 10 | 69 | M | T2 N2 M0 | Squamous cell carcinoma, Right upper lobe, |
| | | | | Stage 3A, tumor size = 9 × 8 × 8 |
| 11 | 66 | M | T2 N0 M0 | Squamous cell carcinoma poorly diff, Right |
| | | | | upper lobe lobectomy, Stage 1B, tumor size = |
| | | | | 2.2 × 1.5 × 1.5 |
| 12 | 51 | M | T2 N2 M0 | Squamous cell carcinoma, Left |
| | | | | pneumonectomy, Stage 3A, tumor size = |
| | | | | 4 × 4 × 3.5 |

A panel of 15 amplification targets was chosen for real-time RT-PCR quantification based on the results of microarray expression analyses (mirVana™ miRNA Bioarray V1, Ambion) performed with normal human lung total RNA (Ambion). The 15 target miRNAs are both highly and uniformly expressed in normal human lung. Array analyses were performed as described (Shingara et al, 2005). The panel of targets included hsa-let-7a, hsa-miR-16, hsa-miR-17-5p, hsa-miR-24, hsa-miR-27a, hsa-miR-30d, hsa-miR-93, hsa-miR-103, hsa-miR-106a, hsa-miR-125a, hsa-miR-143, hsa-miR-146a, hsa-miR-191, hsa-miR-195, and hsa-miR-221. Because of its historical reference as a normalization target, 5S rRNA was also included as an amplification target. Reverse transcription (RT) reaction components were assembled on ice prior to the addition of RNA template (Table 2). Next, 500 picograms of total RNA template were added and mixed. RT reactions were incubated in a 384-well GeneAmp® PCR System 9700 (Applied Biosystems) at 16° C. for 30 minutes, 42° C. for 30 minutes, 85° C. for 5 minutes, then placed on wet ice.

TABLE 2
Reverse Transcription reaction components.

| | μl per | Final | |
|---|---|---|---|
| Component | 10 μl rxn | Concentration | |
| Nuclease-free water | 5.30 | | |
| 10X Reverse Transcription Buffer | 1.00 | 1X | |
| (Ambion) | | | |
| dNTP mix (2.5 mM each) (Ambion) | 1.00 | 0.25 | mM each |
| 5X RT Primer | 0.50 | 0.25X | |
| RNase Inhibitor (40 U/ul) (Ambion) | 0.10 | 0.4 | U/μl |
| Moloney Murine Leukemia Virus | 0.10 | 1 | U/μl |
| Reverse Transcriptase | | | |
| (MMLV-RT) (100 U/μl) (Ambion) | | | |
| Human Lung Total RNA | 2.00 | | |

For PCRs, reaction components (Table 3) were assembled on ice prior to the addition of the cDNA from the RT reactions. Following assembly of the PCR reaction components, 2 μl of the corresponding RT reaction were transferred to the PCR mix. PCRs were incubated in an ABI PRISM™ 7900HT Fast Real-Time PCR system (Applied Biosystems) at 95° C. for 1 minute, then for 40 cycles at 95° C. for 5 seconds and 60° C. for 30 seconds. Results were analyzed with SDS V2.3 (Applied Biosystems).

TABLE 3
Real-time PCR components.

| | μl per | Final | |
|---|---|---|---|
| Component | 15 μl rxn | Concentration | |
| Nuclease-free water | 7.80 | | |
| MgCl₂ (50 mM) | 1.50 | 5 | mM |
| 10X Platinum PCR Buffer, Minus | 1.50 | 1X | |
| Mg (Invitrogen Corp., Carlsbad, CA, | | | |
| USA) | | | |
| dNTP mix (2.5 mM each) (Ambion) | 1.50 | 0.25 | nM each |
| 20X TaqMan Assay | 0.30 | 0.4X | |
| 50X ROX Internal marker (Invitrogen) | 0.30 | 1X | |
| Platinum ® Taq DNA Polymerase | 0.10 | 0.033 | U/μl |
| (5 U/μl) (Invitrogen) | | | |
| cDNA from RT reaction | 2.00 | | |

Using the algorithm software tool NormFinder (Andersen et al., 2004) the inventors determined that hsa-let-7 and hsa-miR-103 were the most suitable targets for normalization with the targets of interest and this tissue sample set. For normalization of the real-time RT-PCR results, the cycle threshold (Cₜ) value (a log value) for the microRNA of interest was subtracted from the geometric mean Cₜ value of hsa-let-7a and hsa-miR-103. In this case, the fold change was determined by subtracting the dCₜ NAT from the corresponding dCₜ Tumor, producing a ddCₜ(T−N) value for each sample. The average ddCₜ(T−N) value across all samples was converted to fold change by 2^{ddCt}. The representative p-values were determined by a two-tailed paired Student's t-test from the dCₜ values of tumor and NAT. Fold changes and p-values for the 15 microRNA panel are shown in Table 4.

TABLE 4
Average expression changes and associated p-values of 15
microRNAs in twelve human lung tumor samples compared to
expression in twelve normal adjacent tissue samples. Fold
change; negative values indicate reduced expression in lung
tumor samples and positive values indicate increased
expression in lung tumor samples.

| miRNA | Fold Change^{a} | p-value |
|---|---|---|
| miR-30d | −2.581 | 0.0027 |
| miR-195 | −1.754 | 0.0006 |
| miR-143 | −1.751 | 0.0128 |
| miR-16 | −1.576 | 0.0042 |
| miR-146a | −1.093 | 0.8049 |
| miR-191 | −1.034 | 0.7657 |
| miR-125a | 1.037 | 0.7760 |
| miR-27a | 1.209 | 0.0884 |
| miR-24 | 1.230 | 0.0191 |
| miR-17-5p | 1.242 | 0.1277 |
| miR-106a | 1.394 | 0.0861 |
| miR-93 | 1.674 | 0.0060 |
| 5S rRNA | 1.710 | 0.0187 |
| miR-221 | 2.200 | 0.0003 |
| let-7a | −1.398 | 0.0071 |
| miR-103 | 1.214 | 0.0563 |

^{a}Normalized to geometric mean of let-7a and miR-103. Values for let-7a and miR-103 were normalized to geometric mean of miR-191 and miR-24, which were the most stable pair when let-7a was removed from the analysis.

### Example 2

### hsa-miR-221 is a Biomarker for Human Lung Cancer

To assess the expression of hsa-miR-221 in human lung tumors, real-time RT-PCR was performed as described in Example 1. Normalization of the real-time RT-PCR results to the geometric mean of hsa-let-7a and hsa-miR-103 showed that the expression of hsa-miR-221 is up-regulated an average of 2.2 fold (p-value=0.0003) in 11 of 12 lung tumor samples compared to the normal adjacent tissue samples (Table 5). These results indicate that hsa-miR-221 can be used as a biomarker in a clinical diagnostic assay for distinguishing cancerous from non-cancerous human lung tissue.

TABLE 5
Fold change of hsa-miR-221 expression for each lung
tumor/normal adjacent tissue pair.

| Tumor/NAT Pair | Fold Change^{a} |
|---|---|
| 1 | 2.595 |
| 2 | 1.023 |
| 3 | −1.103 |
| 4 | 1.285 |
| 5 | 2.770 |
| 6 | 1.710 |
| 7 | 3.889 |
| 8 | 3.489 |
| 9 | 1.731 |
| 10 | 2.557 |
| 11 | 3.468 |
| 12 | 4.205 |

^{a}Normalized to geometric mean of let-7a and miR-103.

### Example 3

### hsa-miR-195 is a Biomarker for Human Lung Cancer

To assess the expression of hsa-miR-195 in human lung tumors, real-time RT-PCR was performed as described in Example 1. Normalization of the real-time RT-PCR results to the geometric mean of hsa-let-7a and hsa-miR-103 showed that the expression of hsa-miR-195 is down-regulated an average of 1.754 fold (p-value=0.0006) in 11 of 12 lung tumor samples compared to the normal adjacent tissue samples (Table 6). These results indicate that hsa-miR-195 can be used as a biomarker in a clinical diagnostic assay for distinguishing cancerous from non-cancerous human lung tissue.

TABLE 6
Fold change of hsa-miR-195 expression for each lung tumor/normal
adjacent tissue pair.

| Tumor/NAT Pair | Fold Change^{a} |
|---|---|
| 1 | 1.064 |
| 2 | −1.629 |
| 3 | −2.124 |
| 4 | −1.876 |
| 5 | −1.387 |
| 6 | −1.125 |
| 7 | −1.628 |
| 8 | −1.469 |
| 9 | −3.378 |
| 10 | −4.044 |
| 11 | −1.536 |
| 12 | −1.773 |

^{a}Normalized to geometric mean of let-7a and miR-103.

### Example 4

### hsa-miR-93 is a Biomarker for Human Lung Cancer

To assess the expression of hsa-miR-93 in human lung tumors, real-time RT-PCR was performed as described in Example 1. Normalization of the real-time RT-PCR results to the geometric mean of hsa-let-7a and hsa-miR-103 showed that the expression of hsa-miR-93 is up-regulated an average of 1.674 fold (p-value=0.006) in 10 of 12 lung tumor samples compared to the normal adjacent tissue samples (Table 7). These results indicate that hsa-miR-93 can be used as a biomarker in a clinical diagnostic assay for distinguishing cancerous from non-cancerous human lung tissue.

TABLE 7
Fold change of hsa-miR-93 expression for each lung tumor/normal
adjacent tissue pair.

| Tumor/NAT Pair | Fold Change^{a} |
|---|---|
| 1 | 1.684 |
| 2 | 3.037 |
| 3 | 2.215 |
| 4 | −1.054 |
| 5 | 3.276 |
| 6 | 3.367 |
| 7 | 1.032 |
| 8 | −1.180 |
| 9 | 1.083 |
| 10 | 2.688 |
| 11 | 1.037 |
| 12 | 1.550 |

^{a}Normalized to geometric mean of let-7a and miR-103.

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,287
U.S. Pat. No. 5,610,289
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,154
U.S. Pat. No. 5,714,606
U.S. Pat. No. 5,728,525
U.S. Pat. No. 5,744,305
U.S. Pat. No. 5,763,167
U.S. Pat. No. 5,777,092
U.S. Pat. No. 5,792,847
U.S. Pat. No. 5,800,992
U.S. Pat. No. 5,807,522
U.S. Pat. No. 5,830,645
U.S. Pat. No. 5,837,196
U.S. Pat. No. 5,847,219
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 6,004,755
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. patent application Ser. No. 09/545,207
U.S. patent application Ser. No. 10/667,126
U.S. patent application Ser. No. 11/141,707
U.S. patent application Ser. No. 11/273,640
U.S. Patent Prov. Appn. Ser. No. 60/575,743
U.S. Patent Prov. Appn. Ser. No. 60/649,584
U.S. Patent Prov. Appn. Ser. No. 60/826,173
Andersen et al., Cancer Res., 64(15):5245-5250, 2004.
Austin-Ward and Villaseca, Revista Medica de Chile, 126(7):838-845, 1998.
Aznar et al., FEMS Immunol. Med. Microbiol., 18(1):31-37, 1997.
Bagga et al., Cell, 122(4):553-563, 2005.
Bukowski et al., Clinical Cancer Res., 4(10):2337-2347, 1998.
Calin and Croce, Nat. Rev. Cancer, 6(11):857-866, 2006.
Carrington et al. Science, 301(5631):336-338, 2003.
Christodoulides et al., Microbiology, 144(Pt 11):3027-3037, 1998.
Cummins et al., In: IRT. Nucleosides and nucleosides, La Jolla Calif., 72, 1996.
Davidson et al., J. Immunother., 21(5):389-398, 1998.
Denli et al., Trends Biochem. Sci., 28:196, 2003.
Didenko, Biotechniques, 31(5):1106-16, 1118, 1120-1, 2001.
Emptage et al., Neuron, 2001 January; 29(1):197-208, 2001.
EP 266,032
EP 373 203
EP 785 280
EP 799 897
Esquela-Kerscher and Slack, Nat. Rev. Cancer, 6(4):259-269, 2006.
Froehler et al., Nucleic Acids Res., 14(13):5399-5407, 1986.
Galardi et al., J. Biol. Chem., 282(32):23716-23724, 2007.
Griffey et al., J Mass Spectrom, 32(3):305-13, 1997.
Hanibuchi et al., Int. J. Cancer, 78(4):480-485, 1998.
Hellstrand et al., Acta Oncologica, 37(4):347-353, 1998.
Hui and Hashimoto, Infection Immun., 66(11):5329-5336, 1998.
Itakura and Riggs, Science, 209:1401-1405, 1980.
Jemal et al., CA Cancer J. Clin., 57:43-66, 2007.
Klostermeier and Millar, Biopolymers, 61(3):159-79, 2001-2002.
Lagos-Quintana et al., Science, 294(5543):853-858, 2001.
Lau et al., Science, 294(5543):858-862, 2001.
Lee and Ambros, Science, 294(5543):862-864, 2001.
Lim et al., Nature, 433(7027):769-773, 2005.
Molina et al., Int. J. Biol. Markers, 9(2):96-101, 1994.
Olsen et al., Dev. Biol., 216:671, 1999.
PCT Appln. WO 0138580
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
Pietras et al., Oncogene, 17(17):2235-2249, 1998.
Remington's Pharmaceutical Sciences” 15th Edition, pages 1035-1038 and 1570-1580, 1990.
Sambrook et al., In: DNA microarrays: a molecular cloning manual, Cold Spring Harbor Laboratory Press, Cold Spring Harbor, N.Y., 2003.
Sambrook et al., In: Molecular cloning: a laboratory manual, 2^{nd} Ed., Cold Spring Harbor Laboratory Press, Cold Spring Harbor, N.Y., 1989.
Sambrook et al., In: Molecular cloning: a laboratory manual, 3^{rd} Ed., Cold Spring Harbor Laboratory Press, Cold Spring Harbor, N.Y., 2001.
Seggerson et al., Dev. Biol., 243:215, 2002.
Shingara J., et al., RNA 11: 1461-1470, 2005.
U.K. Patent 1,529,202
U.K. Patent 8 803 000
Volinia et al., Proc. Natl. Acad. Sci. USA, 103(7):2257-2261, 2006.
Yanaihara et al., Cancer Cell, 9:189-198, 2006.

1. A method for diagnosing lung cancer in a patient comprising evaluating expression of miR-221 and/or miR-195 in one or more samples from the patient, wherein a difference in the expression in the sample from the patient and expression in a normal sample or reference is indicative of lung cancer.

2. The method of claim 1, further comprising evaluating expression of miR-93.

3. The method of claim 1, wherein the miRNA is miR-221.

4. The method of claim 3, further comprising evaluating the expression of miR-93

5. The method of claim 4, further comprising evaluating the expression of miR-195.

6. The method of claim 1, wherein an increase in expression of miR-93, miR-221, or both miR-93 and miR-221 in a patient sample is indicative of lung cancer.

7. The method of claim 1, wherein a decrease in expression of miR-195 in a patient sample is indicative of lung cancer.

8. The method of claim 1, wherein the lung cancer is non-small cell lung cancer, small cell lung cancer, squamous cell carcinoma, adenocarcinoma, large cell carcinoma, adenosquamous carcinoma, or undifferentiated carcinoma.

9. The method of claim 1, further comprising evaluating expression of one or more mRNA whose expression level is correlated with lung cancer.

10. The method of claim 9, wherein the mRNA measured is selected from the group comprising hnRNPB1, telomerase catalytic component (hTERT), EGFR, RAP1, survivin, osteopontin, CYFRA 21.1, and CEA.

11. (canceled)

12. The method of claim 1, wherein the sample is tissue, blood, serum, urine, sputum, or plasma sample.

13. The method of claim 12, wherein the sample is fresh, frozen, fixed, or embedded.

14. The method of claim 1, wherein the sample from the patient and the normal sample are lung samples.

15. The method of claim 1, further comprising obtaining a sample from the patient.

16. (canceled)

17. The method of claim 1, wherein expression of the miRNA is determined by an amplification assay or a hybridization assay.

18. (canceled)

19. The method of claim 17, wherein the quantitative amplification assay is quantitative RT-PCR.

20. (canceled)

21. The method of claim 1, wherein diagnosing is screening for a pathological condition, assessing prognosis of a pathological condition, staging a pathological condition, or assessing response of a pathological condition to therapy.

22. The method of claim 1, further comprising providing a report of the evaluation.

23. A method of treating a patient comprising obtaining a measure of expression for miR-221 and/or miR-195 and selecting treatment based on the measure of miR-221 or miR-195 expression.

24-29. (canceled)

30. A kit for analysis of a sample by assessing miRNA profile for a sample comprising, in suitable container means, two or more miRNA hybridization or amplification reagents comprising one or more probe or amplification primer for miR-221, miR-93, or miR-195.

31-33. (canceled)


SEQUENCE LISTING (45 sequences)

SEQ ID NO: 1 - RNA, 110 nt, Artificial Sequence
  Synthetic primer
ugaacauccaggucuggggcaugaaccuggcauacaauguagauuucuguguucguuaggcaacagcuacauugucugcuggguuucaggcuaccuggaaacauguucuc

SEQ ID NO: 2 - RNA, 23 nt, Artificial Sequence
  Synthetic primer
agcuacauugucugcuggguuuc

SEQ ID NO: 3 - RNA, 27 nt, Artificial Sequence
  Synthetic primer
agcuacaucugugcugacuggguucuc

SEQ ID NO: 4 - RNA, 80 nt, Artificial Sequence
  Synthetic primer
cugggggcuccaaagugcuguucgugcagguagugugauuacccaaccuacugcugagcuagcacuucccgagcccccgg

SEQ ID NO: 5 - RNA, 23 nt, Artificial Sequence
  Synthetic primer
caaagugcuguucgugcagguag

SEQ ID NO: 6 - RNA, 36 nt, Artificial Sequence
  Synthetic primer
caacaagcugcuguaugcguuagcagcguuacgccg

SEQ ID NO: 7 - RNA, 87 nt, Artificial Sequence
  Synthetic primer
agcuucccuggcucuagcagcacagaaauauuggcacagggaagcgagucugccaauauuggcugugcugcuccaggcaggguggug

SEQ ID NO: 8 - RNA, 21 nt, Artificial Sequence
  Synthetic primer
uagcagcacagaaauauuggc

SEQ ID NO: 9 - RNA, 22 nt, Artificial Sequence
  Synthetic primer
uagcagcacagaaauauuggca

SEQ ID NO: 10 - RNA, 80 nt, Artificial Sequence
  Synthetic primer
ugggaugagguaguagguuguauaguuuuagggucacacccaccacugggagauaacuauacaaucuacugucuuuccua

SEQ ID NO: 11 - RNA, 22 nt, Artificial Sequence
  Synthetic primer
ugagguaguagguuguauaguu

SEQ ID NO: 12 - RNA, 33 nt, Artificial Sequence
  Synthetic primer
uagagguaguaugaguauugucaaguuaugguu

SEQ ID NO: 13 - RNA, 89 nt, Artificial Sequence
  Synthetic primer
gucagcagugccuuagcagcacguaaauauuggcguuaagauucuaaaauuaucuccaguauuaacugugcugcugaaguaagguugac

SEQ ID NO: 14 - RNA, 22 nt, Artificial Sequence
  Synthetic primer
uagcagcacguaaauauuggcg

SEQ ID NO: 15 - RNA, 27 nt, Artificial Sequence
  Synthetic primer
uagcagcacuguaaauauuggcguagu

SEQ ID NO: 16 - RNA, 84 nt, Artificial Sequence
  Synthetic primer
gucagaauaaugucaaagugcuuacagugcagguagugauaugugcaucuacugcagugaaggcacuuguagcauuauggugac

SEQ ID NO: 17 - RNA, 23 nt, Artificial Sequence
  Synthetic primer
caaagugcuuacagugcagguag

SEQ ID NO: 18 - RNA, 24 nt, Artificial Sequence
  Synthetic primer
caaagugcuuacagugcagguagu

SEQ ID NO: 19 - RNA, 68 nt, Artificial Sequence
  Synthetic primer
cuccggugccuacugagcugauaucaguucucauuuuacacacuggcucaguucagcaggaacaggag

SEQ ID NO: 20 - RNA, 22 nt, Artificial Sequence
  Synthetic primer
uggcucaguucagcaggaacag

SEQ ID NO: 21 - RNA, 22 nt, Artificial Sequence
  Synthetic primer
uggcucaguucagcaggaacag

SEQ ID NO: 22 - RNA, 78 nt, Artificial Sequence
  Synthetic primer
cugaggagcagggcuuagcugcuugugagcaggguccacaccaagucguguucacaguggcuaaguuccgccccccag

SEQ ID NO: 23 - RNA, 21 nt, Artificial Sequence
  Synthetic primer
uucacaguggcuaaguuccgc

SEQ ID NO: 24 - RNA, 31 nt, Artificial Sequence
  Synthetic primer
uucacaguggcuuaaguuccaugaucucuga

SEQ ID NO: 25 - RNA, 70 nt, Artificial Sequence
  Synthetic primer
guuguuguaaacauccccgacuggaagcuguaagacacagcuaagcuuucagucagauguuugcugcuac

SEQ ID NO: 26 - RNA, 22 nt, Artificial Sequence
  Synthetic primer
uguaaacauccccgacuggaag

SEQ ID NO: 27 - RNA, 25 nt, Artificial Sequence
  Synthetic primer
uguaaacaucucccgacuggaagcu

SEQ ID NO: 28 - RNA, 78 nt, Artificial Sequence
  Synthetic primer
uacugcccucggcuucuuuacagugcugccuuguugcauauggaucaagcagcauuguacagggcuaugaaggcauug

SEQ ID NO: 29 - RNA, 23 nt, Artificial Sequence
  Synthetic primer
agcagcauuguacagggcuauga

SEQ ID NO: 30 - RNA, 24 nt, Artificial Sequence
  Synthetic primer
agcagcauuguacagggcuaugca

SEQ ID NO: 31 - RNA, 81 nt, Artificial Sequence
  Synthetic primer
ccuuggccauguaaaagugcuuacagugcagguagcuuuuugagaucuacugcaauguaagcacuucuuacauuaccaugg

SEQ ID NO: 32 - RNA, 23 nt, Artificial Sequence
  Synthetic primer
aaaagugcuuacagugcagguag

SEQ ID NO: 33 - RNA, 24 nt, Artificial Sequence
  Synthetic primer
aaaagugcuuacagugcagguagc

SEQ ID NO: 34 - RNA, 86 nt, Artificial Sequence
  Synthetic primer
ugccagucucuaggucccugagacccuuuaaccugugaggacauccagggucacaggugagguucuugggagccuggcgucuggcc

SEQ ID NO: 35 - RNA, 24 nt, Artificial Sequence
  Synthetic primer
ucccugagacccuuuaaccuguga

SEQ ID NO: 36 - RNA, 24 nt, Artificial Sequence
  Synthetic primer
ucccugagacccuuuaaccuguga

SEQ ID NO: 37 - RNA, 106 nt, Artificial Sequence
  Synthetic primer
gcgcagcgcccugucucccagccugaggugcagugcugcaucucuggucaguugggagucugagaugaagcacuguagcucaggaagagagaaguuguucugcagc

SEQ ID NO: 38 - RNA, 21 nt, Artificial Sequence
  Synthetic primer
ugagaugaagcacuguagcuc

SEQ ID NO: 39 - RNA, 23 nt, Artificial Sequence
  Synthetic primer
ugagaugaagcacuguagcucga

SEQ ID NO: 40 - RNA, 99 nt, Artificial Sequence
  Synthetic primer
ccgauguguauccucagcuuugagaacugaauuccauggguugugucagugucagaccucugaaauucaguucuucagcugggauaucucugucaucgu

SEQ ID NO: 41 - RNA, 22 nt, Artificial Sequence
  Synthetic primer
ugagaacugaauuccauggguu

SEQ ID NO: 42 - RNA, 28 nt, Artificial Sequence
  Synthetic primer
ugagaacugaauuccauagaggaucugg

SEQ ID NO: 43 - RNA, 92 nt, Artificial Sequence
  Synthetic primer
cggcuggacagcgggcaacggaaucccaaaagcagcuguugucuccagagcauuccagcugcgcuuggauuucguccccugcucuccugccu

SEQ ID NO: 44 - RNA, 23 nt, Artificial Sequence
  Synthetic primer
caacggaaucccaaaagcagcug

SEQ ID NO: 45 - RNA, 23 nt, Artificial Sequence
  Synthetic primer
caacggaaucccaaaagcagcug